(19) United States
(12) Patent Application Publication
(10) Pub. No.: US 2009/0171152 A1
(43) Pub. Date: Jul. 2, 2009

(54) Insertion assisting instrument for medical device

(76) Inventors: Isao Aoki, Kanagawa (JP); Hironobu Takizawa, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/630,449

(22) PCT Filed: May 19, 2006
(86) PCT No.: PCT/JP2006/310050

§ 371 Date: Dec. 21, 2006

(30) Foreign Application Priority Data
Jul. 28, 2005 (JP) 2005-219469

(51) Int. Cl. A61B 1/01

(52) U.S. Cl. 600/114

(57) ABSTRACT

An endoscope insertion assisting instrument 7 as an insertion assisting instrument for a medical device of the present invention comprises an elongated assisting instrument insertion portion 31 having flexibility for guiding an endoscope 2, which is the medical device, into a deep portion in an intracoelomic duct, a helical structure portion 33 provided on the outer circumference of the assisting instrument insertion portion 31, and a plurality of balloons 32 as locking portions for holding at least a part of the shape of the intracoelomic duct into which the endoscope 2 is inserted and for locking the assisting instrument insertion portion 31 in the intracoelomic duct.

By holding at least a part of the shape of the intracoelomic duct into which a medical device is inserted at insertion of the medical device into the deep portion of the intracoelomic duct by the insertion assisting instrument for a medical device of the present invention, excessive deformation of the intracoelomic duct is suppressed and insertion performance of the medical device into the deep portion of the intracoelomic duct is improved.

### TECHNICAL FIELD

The present invention relates to an insertion assisting instrument for a medical device assisting insertion of the medical device such as an endoscope into an intracoelomic duct of a subject.

### BACKGROUND ART

An endoscope has been widely used in the medical field or the like. The endoscope has an insertion portion so as to be inserted into a body cavity to perform inspection, observation, treatment and the like inside the body cavity.

When a medical device such as an endoscope having this type of insertion portion is to be used, such an insertion assisting instrument for a medical device is proposed and is designed so that the insertion portion can be smoothly inserted into a bent portion of the intracoelomic duct.

As an example, Japanese Patent Laid-Open No. 1-203704 discloses an actuator as the insertion assisting instrument for the medical device. For the actuator, two balloons of a traveling instrument provided at the tip end portion of the endoscope insertion portion are alternatively inflated/deflated. By this, the actuator expands/contracts a cylindrical member arranged between the two balloons to drive ahead the endoscope insertion portion into the deep portion in the body cavity.

Japanese Patent Laid-Open No. 2004-97391, for example, discloses an endoscope device provided with fixed holding means as the insertion assisting instrument for the medical device. The endoscope device individually inflates two balloons provided at the tip end portion of an endoscope insertion portion in compliance with the intracoelomic duct. Also, the fixed holding means used in the endoscope device has the tip end portion of the endoscope insertion portion locked in the intracoelomic duct. Therefore, the tip end portion of the endoscope insertion portion can be fixed in the intracoelomic duct by the fixed holding means and is also capable of micromotion in the vertical/horizontal direction and elevation angle/azimuth.

Moreover, U.S. Pat. No. 5,989,230 discloses a catheter provided with a helical structure portion as the insertion assisting instrument for the medical device. With regard to the helical structure portion used in the catheter, a thrust that a male screw acts on a female screw by rotation is generated at contact with the intracoelomic duct wall for propelling into the deep portion in the body cavity.

Moreover, as the insertion assisting instrument for the medical device assisting a conventional medical device, a guide wire is used, for example. This guide wire is inserted to a target portion in the intracoelomic duct by being inserted through a treatment instrument insertion channel of the endoscope insertion portion, protruded from a channel opening and locked at the target portion, for example.

When the endoscope insertion portion is being inserted into the target portion along the guide wire, the guide wire as the insertion assisting instrument for the medical device can freely move along the intracoelomic duct which is changeable in shape.

For this reason, as the endoscope insertion portion is inserted into the deep portion in the body cavity along the guide wire, it is concerned that the tip end portion may contact and thereby pressing, e.g., a wall surface of the bent portion of the intracoelomic duct, resulting in excessive deformation of the intracoelomic duct. Such excessive deformation of the intracoelomic duct may cause the guide wire to have a length and shape unsuitable for the intracoelomic duct, thus making it difficult to guide the endoscope insertion portion into the target position of the intracoelomic duct.

The present invention has been made in light of the above-mentioned problems, and aims to provide an insertion assisting instrument for a medical device which suppresses excessive deformation of the intracoelomic duct and improves insertion performance of the medical device into the deep portion of the intracoelomic duct, by holding at least a part of the shape of the intracoelomic duct into which a medical device is inserted when the medical device is inserted into the deep portion of the intracoelomic duct.

### DISCLOSURE OF THE INVENTION

### Means for Solving the Problem

In order to solve the above problem, an insertion assisting instrument for a medical device according to an embodiment of the present invention comprises an elongated insertion portion having flexibility for guiding a medical device into a deep portion in an intracoelomic duct, a helical structure portion provided on the outer circumference of the insertion portion, and a plurality of locking portions for holding at least a part of the shape of the intracoelomic duct into which the medical device is inserted and for locking the insertion portion in the intracoelomic duct.

The insertion assisting instrument for the medical device according to the present invention constructed as above has an effect that by holding at least a part of the shape of the intracoelomic duct into which the medical device is inserted when the medical device is inserted into a deep portion of the intracoelomic duct, excessive deformation of the intracoelomic duct is suppressed and the insertion performance of the medical device into the deep portion of the intracoelomic duct can be improved.

### BRIEF DESCRIPTION OF THE DRAWINGS

FIG. 1 is an entire configuration diagram showing an endoscope device according to a first embodiment;

FIG. 2 is a perspective view showing the tip end side of an insertion portion of the endoscope in FIG. 1;

FIG. 3 is an outline view showing an endoscope insertion assisting instrument and a rotational driving portion of a rotational driving device in FIG. 1;

FIG. 4 is a sectional view showing a fluid supply portion mounted at the assisting instrument insertion portion and the assisting instrument insertion portion;

FIG. 5 is a V-V sectional view of FIG. 4;

FIG. 6 is a sectional view showing the vicinity of a balloon of the assisting instrument insertion portion;

FIG. 7 is an outline view showing a state where the endoscope insertion assisting instrument inserted into a channel for treatment instrument insertion of the endoscope is inserted through an anus into a deep portion side of a large intestine;

FIG. 8 is an outline view showing a state where the endoscope insertion assisting instrument has been inserted to the vicinity of a cecum from the state in FIG. 7;

FIG. 9 is an outline view showing a state where all the balloons of the endoscope insertion assisting instrument are inflated from the state in FIG. 8;

FIG. 10 is an outline view showing a case where a usual guide wire is used to pass the endoscope tip end portion from a rectum to a sigmoid colon portion;

FIG. 11 is an outline view showing a case where the endoscope tip end portion is passed from the rectum to the sigmoid colon portion from the state in FIG. 9;

FIG. 12 is a sectional view showing a variation of the assisting instrument insertion portion provided with a common piping;

FIG. 13 is an outline view showing a variation of the assisting instrument insertion portion provided with a sensor in the vicinity of the balloon;

FIG. 14 is an enlarged view of an essential part in the vicinity of the sensor in FIG. 13;

FIG. 15 is an enlarged view of an essential part in the vicinity of the sensor showing a first variation in FIG. 14;

FIG. 16 is an enlarged view of an essential part in the vicinity of the sensor showing a second variation in FIG. 14;

FIG. 17 is an enlarged view of an essential part in the vicinity of the sensor showing a third variation in FIG. 14;

FIG. 18 is an outline view showing a helical balloon in which a helical structure portion at substantially the same position is formed by a balloon instead of the balloon;

FIG. 19 is an outline view showing a state where the helical balloon in FIG. 18 is inflated;

FIG. 20 is an outline view showing a balloon with a helix provided with the helical structure portion on the outer circumferential surface of the assisting instrument insertion portion;

FIG. 21 is an outline view showing a state where the balloon with a helix in FIG. 20 is inflated;

FIG. 22 is an outline view showing a balloon provided on the outer circumference of the helical structure portion;

FIG. 23 is an outline view showing a state where the balloon in FIG. 22 is inflated;

FIG. 24 is an outline view showing the helical structure portion into which a coil spring is inserted;

FIG. 25 is an outline view showing a state when the coil spring in FIG. 24 is released and inflated;

FIG. 26 is a sectional view of the assisting instrument insertion portion provided with a suction portion instead of the balloon;

FIG. 27 is a sectional view showing a variation of the assisting instrument insertion portion provided with a common suction piping in contrast with FIG. 26;

FIG. 28 is a perspective view showing a construction of a tip end side of the endoscope device according to a second embodiment;

FIG. 29 is a view showing a holding body for propelling in a first variation;

FIG. 30 is a view showing a structure of the holding body for propelling in FIG. 29;

FIG. 31 is a perspective view showing an outline construction of the holding body for propelling in a second variation;

FIG. 32 is a view showing an internal construction of the holding body for propelling in FIG. 31;

FIG. 33 is a perspective view showing the vicinity of the holding body for propelling with the endoscope mounted in a third variation;

FIG. 34 is a perspective view showing an outline construction of the holding body for propelling in FIG. 33;

FIG. 35 is a view showing an internal construction of the holding body for propelling in FIG. 34;

FIG. 36 is a perspective view showing a tip end side of a fourth variation inserted into an exclusive endoscope channel;

FIG. 37 is a perspective view showing the vicinity of the tip end portion of the endoscope in FIG. 36;

FIG. 38 is a front view of the endoscope in FIG. 37; and

FIG. 39 is a perspective view showing a state where the treatment instrument is inserted into a hollow portion of a fourth variation.

### BEST MODE FOR CARRYING OUT THE INVENTION

Embodiments of the present invention will be described below referring to the attached drawings.

In the following embodiments, the present invention is applied to a large intestine endoscope as a medical device.

### FIRST EMBODIMENT

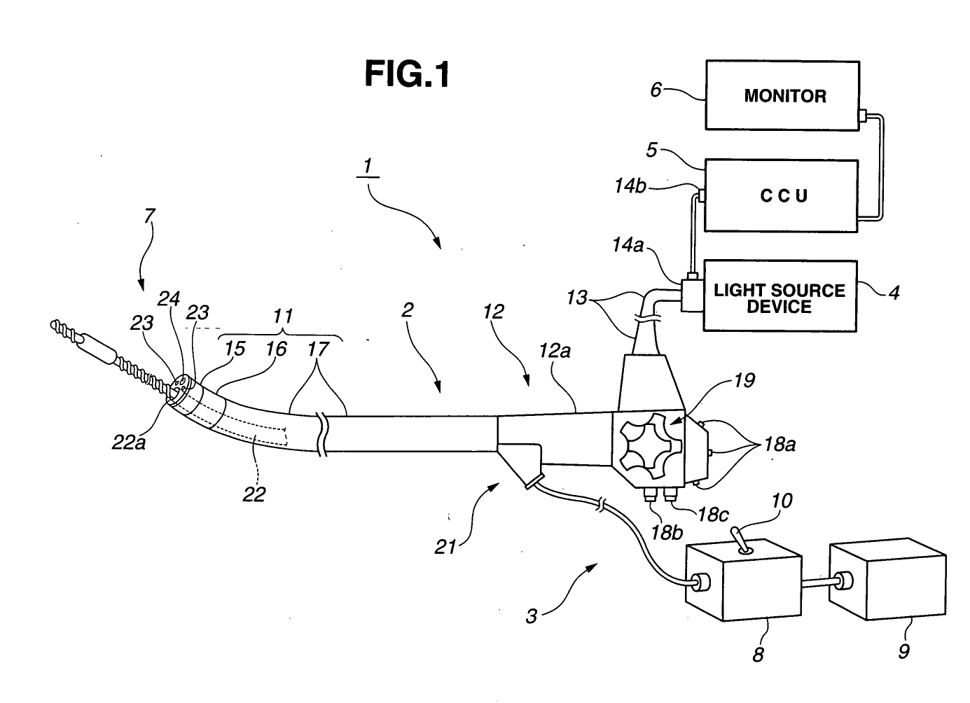

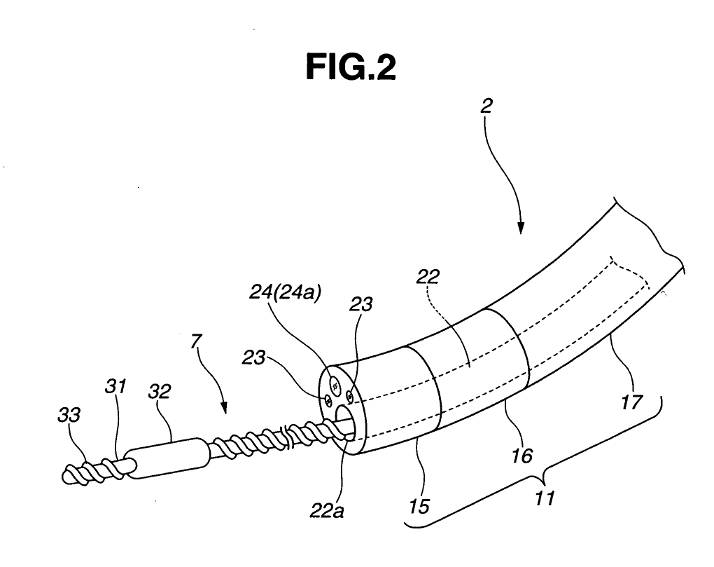

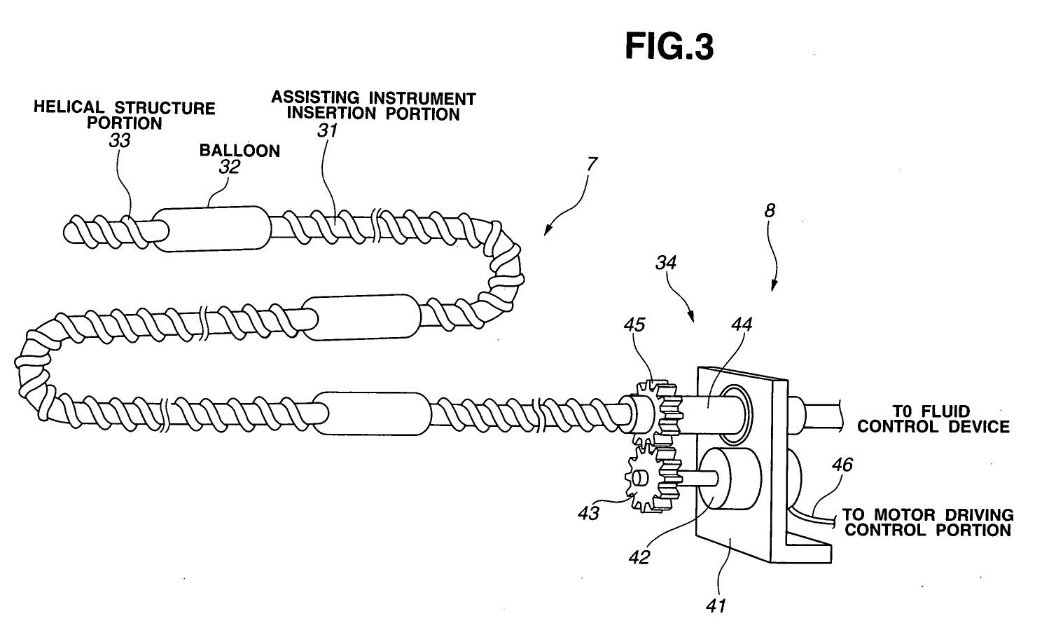

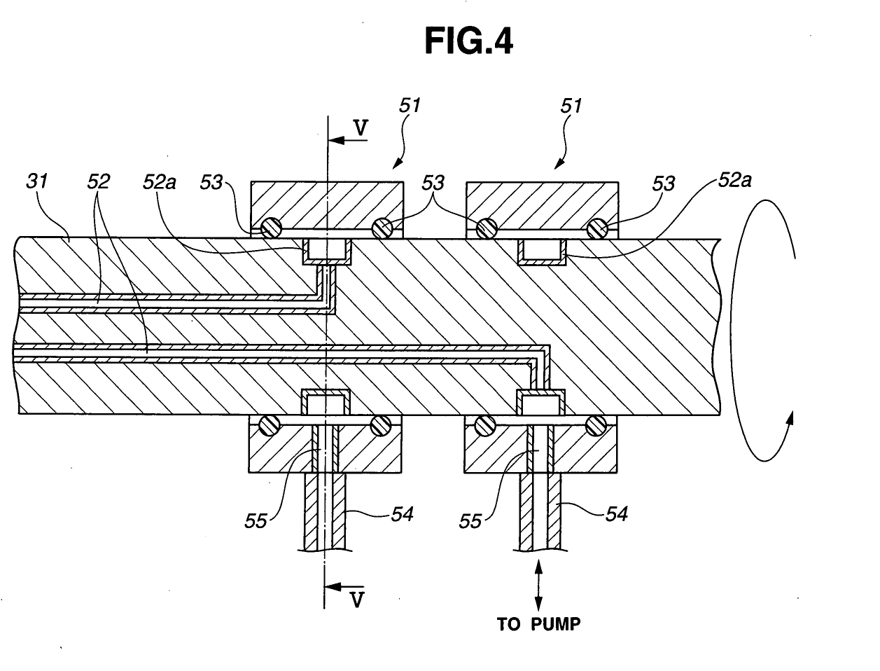

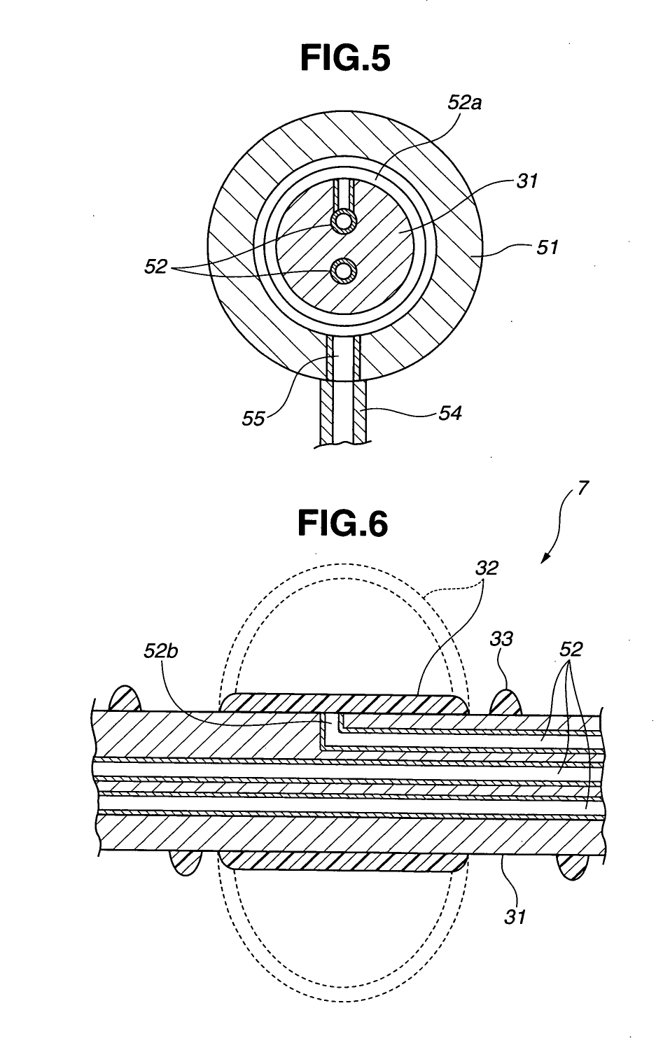

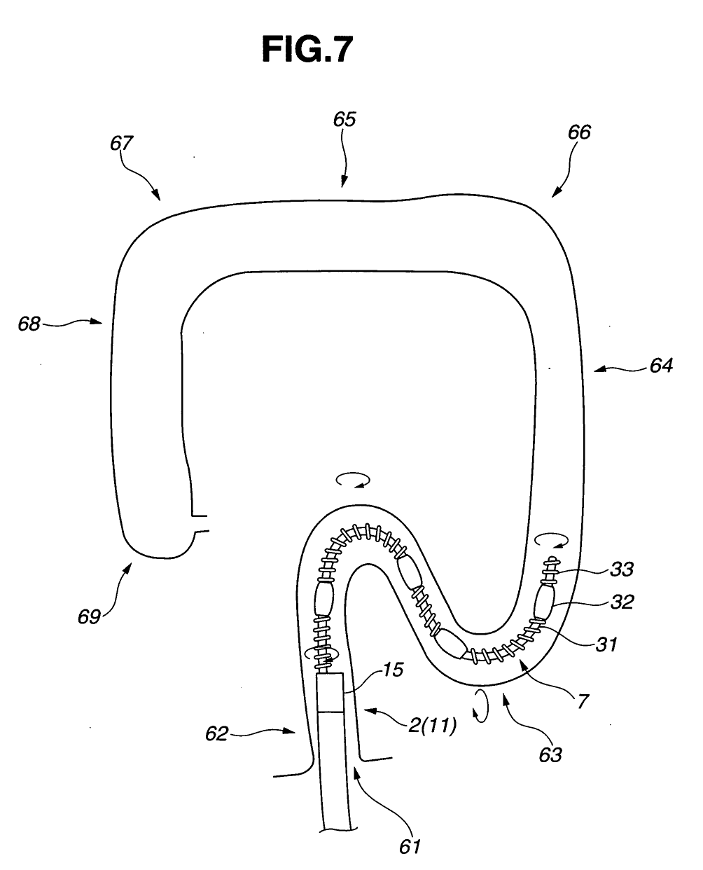

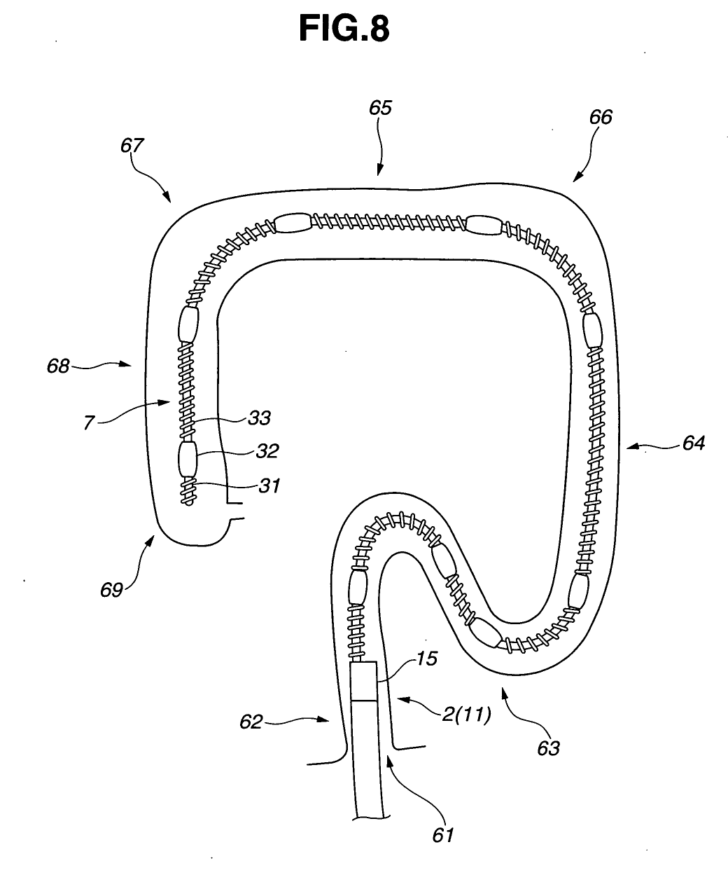

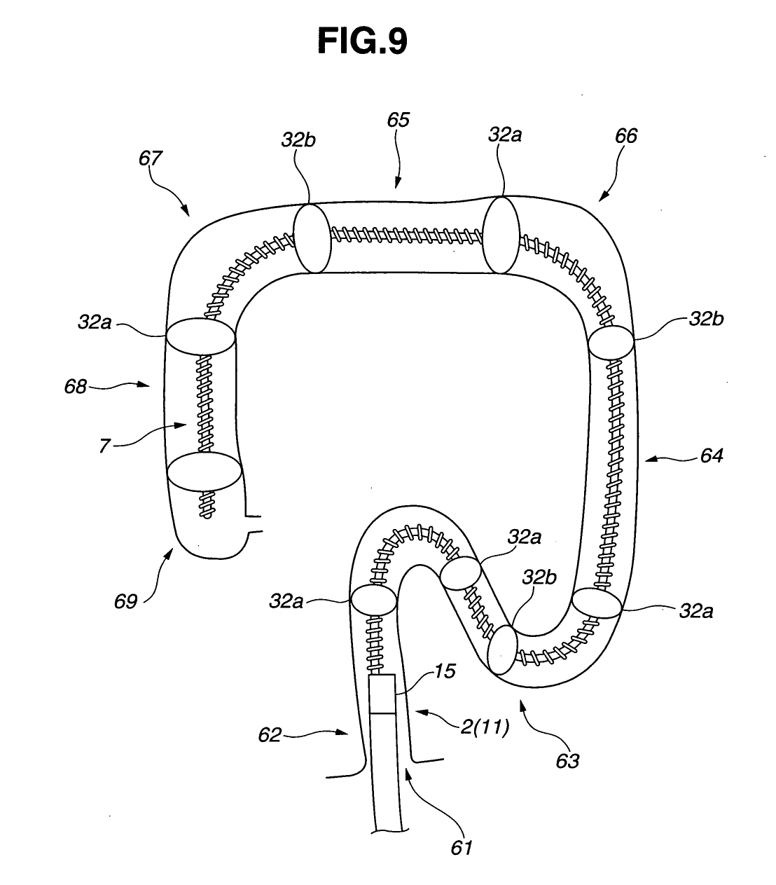

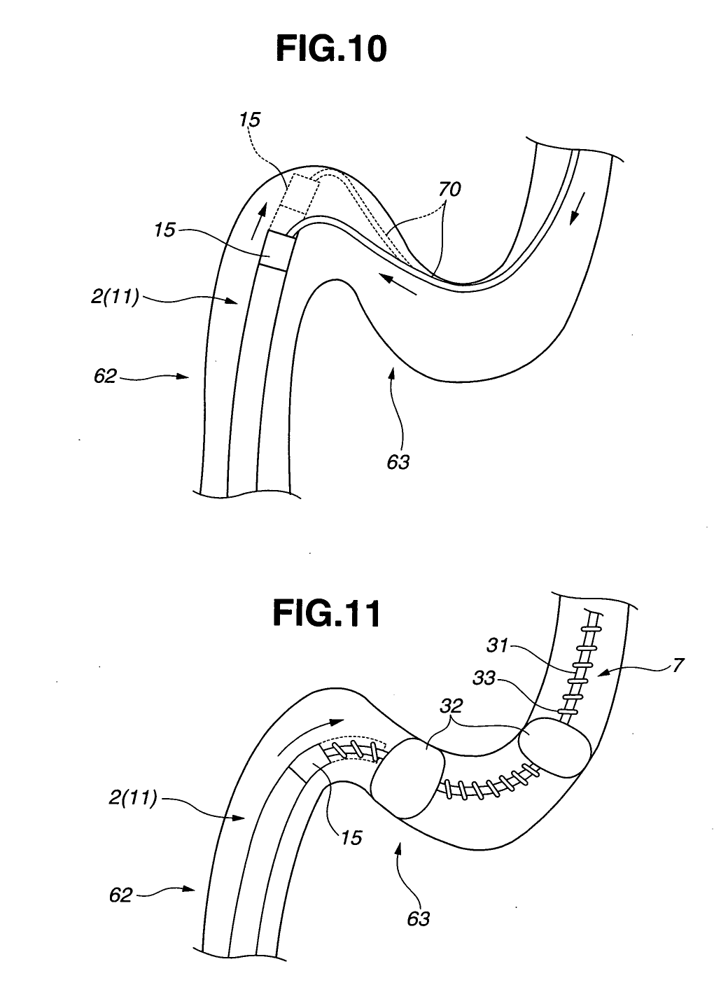

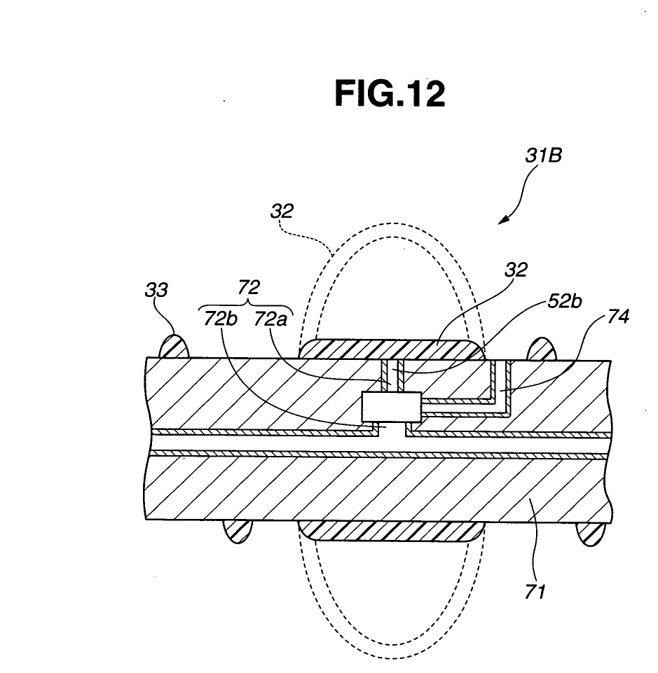

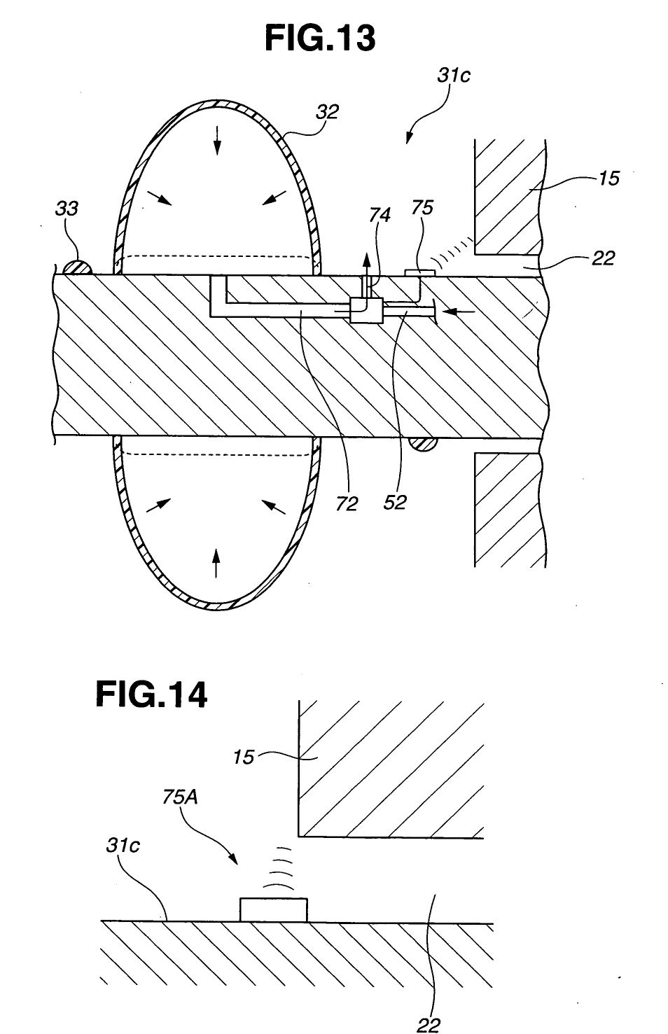

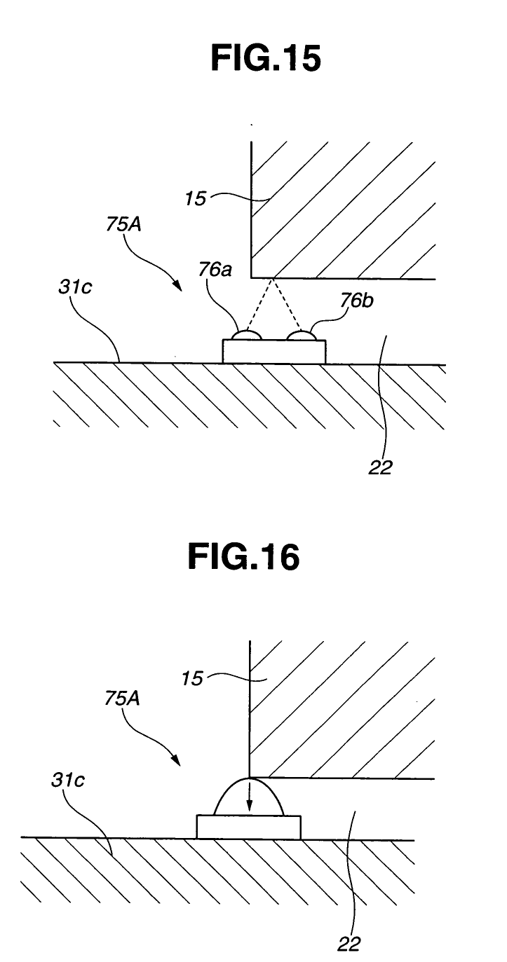

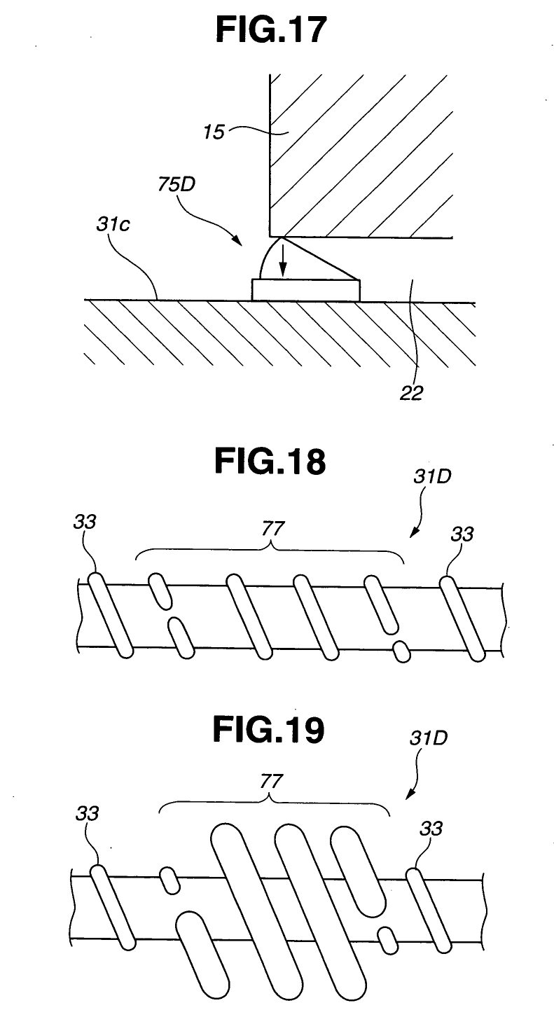

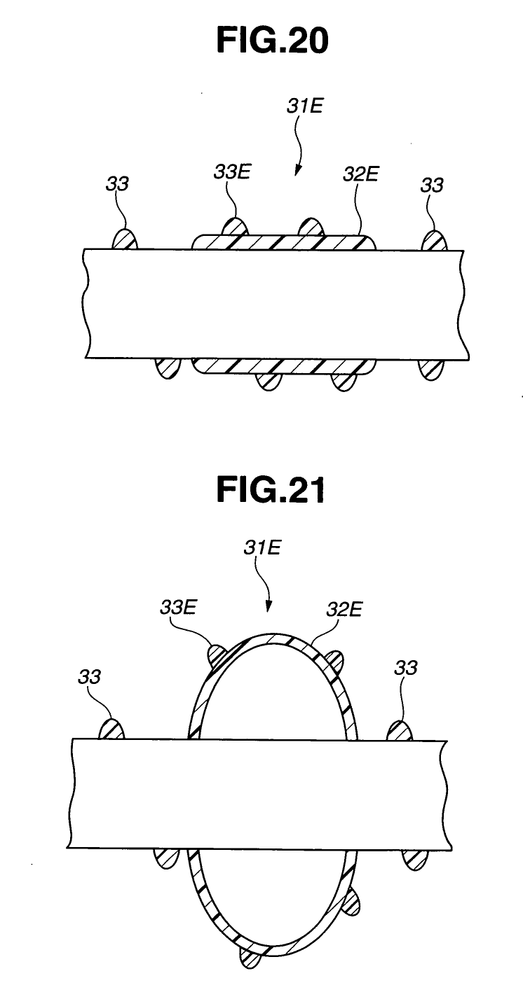

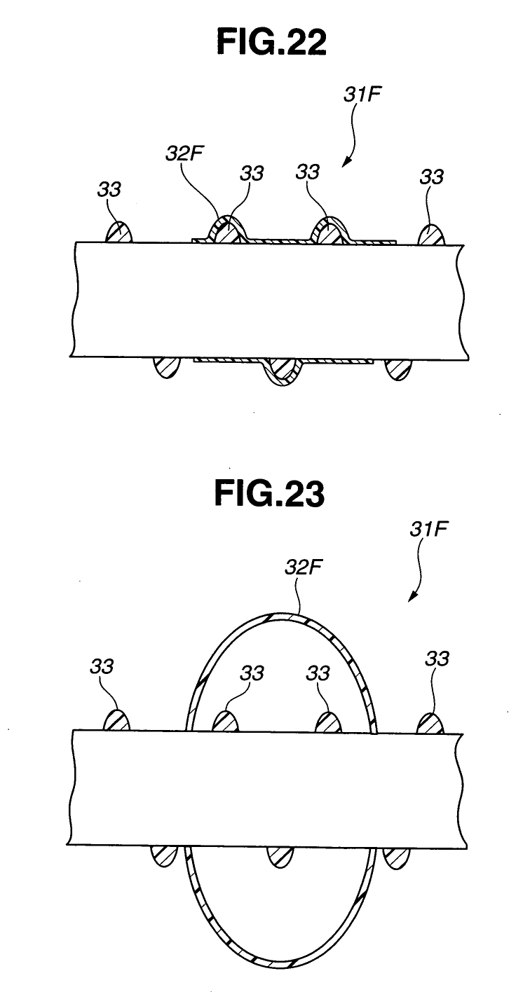

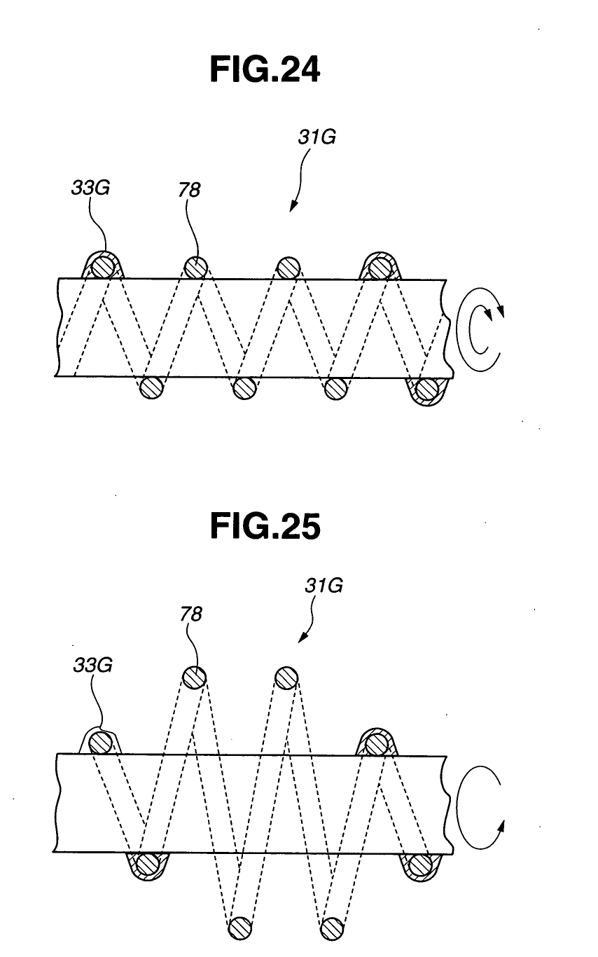

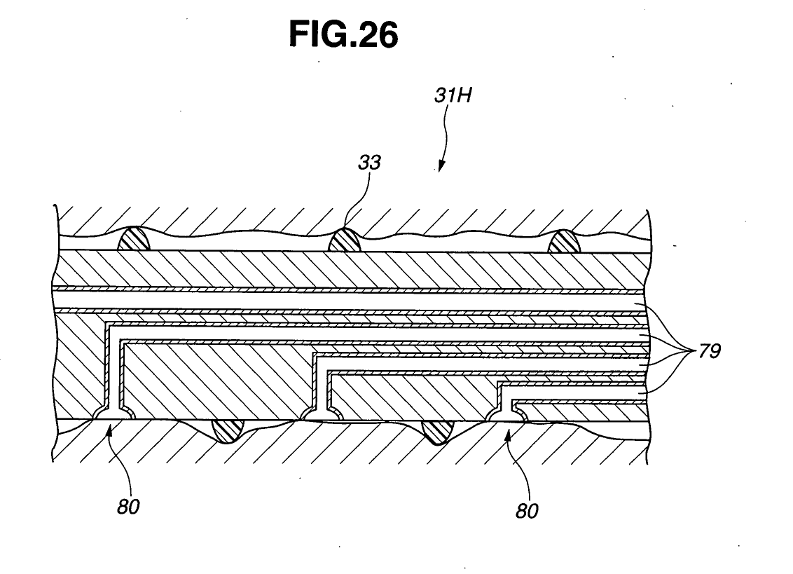

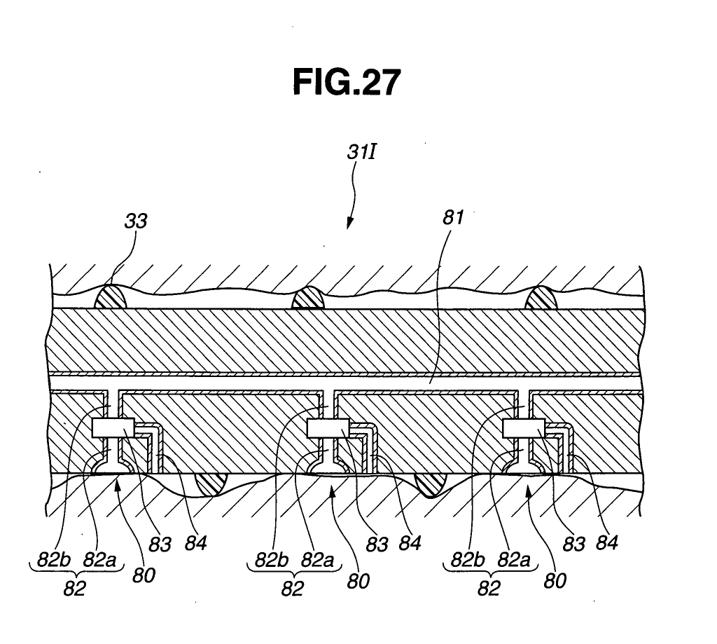

FIGS. 1 to 27 relate to a first embodiment of the present invention, in which FIG. 1 is an entire configuration diagram showing an endoscope device according to a first embodiment, FIG. 2 is a perspective view showing the tip end side of an insertion portion of the endoscope in FIG. 1, FIG. 3 is an outline view showing an endoscope insertion assisting instrument and a rotational driving portion of a rotational driving device in FIG. 1, FIG. 4 is a sectional view showing a fluid supply portion mounted at the assisting instrument insertion portion and the assisting instrument insertion portion, FIG. 5 is a V-V sectional view of FIG. 4, FIG. 6 is a sectional view showing the vicinity of a balloon of the assisting instrument insertion portion, FIG. 7 is an outline view showing a state where the endoscope insertion assisting instrument inserted into a channel for treatment instrument insertion of the endoscope is inserted through an anus into a deep portion side of a large intestine, FIG. 8 is an outline view showing a state where the endoscope insertion assisting instrument has been inserted to the vicinity of a cecum from the state in FIG. 7, FIG. 9 is an outline view showing a state where all the balloons of the endoscope insertion assisting instrument are inflated from the state in FIG. 8, FIG. 10 is an outline view showing a case where a usual guide wire is used to pass the endoscope tip end portion from a rectum to a sigmoid colon portion, FIG. 11 is an outline view showing a case where the endoscope tip end portion is passed from the rectum to the sigmoid colon portion from the state in FIG. 9, FIG. 12 is a sectional view showing a variation of the assisting instrument insertion portion provided with a common piping, FIG. 13 is an outline view showing a variation of the assisting instrument insertion portion provided with a sensor in the vicinity of the balloon, FIG. 14 is an enlarged view of an essential part in the vicinity of the sensor in FIG. 13, FIG. 15 is an enlarged view of an essential part in the vicinity of the sensor showing a first variation in FIG. 14, FIG. 16 is an enlarged view of an essential part in the vicinity of the sensor showing a second variation in FIG. 14, FIG. 17 is an enlarged view of an essential part in the vicinity of the sensor showing a third variation in FIG. 14, FIG. 18 is an outline view showing a helical balloon in which a helical structure portion at substantially the same position is formed by a balloon instead of the balloon, FIG. 19 is an outline view showing a state where the helical balloon in FIG. 18 is inflated, FIG. 20 is an outline view showing a balloon with a helix provided with the helical structure portion on the outer circumferential surface of the assisting instrument insertion portion, FIG. 21 is an outline view showing a state where the balloon with a helix in FIG. 20 is inflated, FIG. 22 is an outline view showing a balloon provided on the outer circumference of the helical structure portion, FIG. 23 is an outline view showing a state where the balloon in FIG. 22 is inflated, FIG. 24 is an outline view showing the helical structure portion into which a coil spring is inserted, FIG. 25 is an outline view showing a state when the coil spring in FIG. 24 is released and inflated, FIG. 26 is a sectional view of the assisting instrument insertion portion provided with a suction portion instead of the balloon, and FIG. 27 is a sectional view showing a variation of the assisting instrument insertion portion provided with a common suction piping in contrast with FIG. 26.

As shown in FIG. 1, an endoscope device 1 of the first embodiment has a large intestine endoscope (hereinafter simply as an endoscope) 2 for performing inspection, observation, treatment and the like in a large intestine as a medical device, an endoscope insertion assisting device 3 for assisting insertion of this endoscope 2, a light source device 4 for supplying illumination light to the endoscope 2, a camera control unit (abbreviated as CCU) 5 for signal processing for an image pickup device built in the endoscope 2, and a monitor 6 for displaying an endoscopic image captured by the image pickup device by input of a video signal outputted from this CCU 5.

The endoscope insertion assisting device 3 has an endoscope insertion assisting instrument 7 as an insertion assisting instrument for a medical device for inserting a treatment instrument insertion channel 22 of the endoscope 2, which will be described later, and guiding the endoscope 2 into a deep portion of the intracoelomic duct, a rotational driving device 8 for rotating this endoscope insertion assisting instrument 7, and a fluid control device 9 for supplying and discharging a fluid such as air, water and the like as control means (control portion) for a balloon 32, which will be described later, provided at the endoscope insertion assisting instrument 7. The fluid control device 9 has a pump, a valve control portion and a control circuit, not shown, built in and controls inflation/deflation of the balloon 32 according to operation of an operator. Also, the rotational driving device 8 is provided with an operation knob 10 on the upper face of the cabinet.

First, the endoscope 2 will be described.

The endoscope 2 comprises an elongated endoscope insertion portion 11 having flexibility and an operation portion 12 continuously provided on the base end side of this endoscope insertion portion 11 and also functioning as a gripping portion 12a. This endoscope 2 has a universal cord 13 extended from the side of the operation portion 12.

In this universal cord 13, a light guide and a signal line, not shown, are inserted and disposed. A connector portion 14a provided at the end portion of this universal cord 13 is connected to the light source device 4, and a connector portion 14b is connected to the CCU 5.

The endoscope insertion portion 11 of the endoscope 2 comprises a rigid endoscope tip end portion 15, a bending portion 16 capable of being bent, and a lengthy flexible pipe portion 17 having flexibility continuously provided. The bending portion 16 is provided at the base end side of the endoscope tip end portion 15. The flexible pipe portion 17 is provided at the base end side of the bending portion 16.

The operation portion 12 of the endoscope 2 has the gripping portion 12a on the base end side. The gripping portion 12a is a portion held and gripped by the operator. On the upper part side of the operation portion 12, a video switch 18a for remote control of the CCU 5 is arranged.

Also, at the operation portion 12, an air/water feed switch 18b for operating an air/water feed operation and a suction switch 18c for operating a suction operation are provided. Also, at the operation portion 12, a bending operation knob 19 is provided. The operator can perform bending operation of the bending portion 16 by gripping the gripping portion 12a and operating the bending operation knob 19.

Moreover, at the operation portion 12, a treatment instrument insertion port 21 for inserting a treatment instrument such as biopsy forceps is provided in the vicinity of the front end of the gripping portion 12a. This treatment instrument insertion port 21 communicates with the treatment instrument insertion channel 22 in its inside.

The operator can perform biopsy or the like by inserting a treatment instrument such as forceps, not shown, into the treatment instrument insertion port 21 so that the tip end side of the treatment instrument is protruded from a channel opening 22a formed at the endoscope tip end portion 15 through the treatment instrument insertion channel 22 inside.

In this embodiment, the operator inserts the endoscope insertion assisting instrument 7 through the treatment instrument insertion channel 22 to have the assisting instrument tip end portion protruded from the channel opening 22a by a predetermined distance and inserted into a body cavity so that it reaches a target portion, to which the endoscope insertion portion 11 of the endoscope 2 is guided.

The endoscope 2 has a light guide, not shown, inserted and disposed in the universal cord 13, the endoscope insertion portion 11, the operation portion 12, and illumination light supplied from the light source device 4 is transmitted by this light guide. The illumination light transmitted from the light guide illuminates a subject such as an affected portion through an illumination optical system 23 arranged at the endoscope tip end portion 15.

The reflected light of the illuminated subject is taken in as a subject image from an objective optical system 24a constituting an image capturing device 24 arranged adjacent to the illumination optical system 23. The taken-in subject image is captured by an image capturing portion, not shown, and photoelectrically converted and then converted to an image capturing signal.

This image capturing signal is transmitted through a signal cable extended from the image capturing portion and outputted to the CCU 5 through the operation portion 12 and the connector portion 14b of the universal cord 13.

The CCU 5 executes signal processing of the image capturing signal from the image capturing portion of the endoscope 2, generates a standard video signal and has the endoscopic image displayed on the monitor 6.

Next, the endoscope insertion assisting instrument 7 will be described.

As shown in FIG. 3, the endoscope insertion assisting instrument 7 has a flexible and elongated assisting instrument insertion portion 31. At predetermined positions of this assisting instrument insertion portion 31, a plurality of balloons 32 formed by an elastic member, for example, are provided as a plurality of locking means (locking portion) to be locked in the intracoelomic duct (See FIG. 9).

Also, on the outer surface of the assisting instrument insertion portion 31, a helical structure portion 33 in which a small-diameter hollow or solid strip-shaped resin is mounted in the helical manner and the portion is protruded in the helical manner from the outer surface are provided. This helical structure portion 33 is formed in the right screw state. The assisting instrument insertion portion 31 can be propelled by being rotated in the clockwise direction.

On the other hand the assisting instrument insertion portion 31 can be moved to the rear side by being rotated in the counterclockwise direction. By this, the endoscope insertion assisting instrument 7 realizes a mechanism for smoothly assisting insertion of the assisting instrument insertion portion 31 into the intracoelomic duct.

At the rear end side of the assisting instrument insertion portion 31, a rotational driving portion 34 of the rotational driving device 8 is provided. This rotational driving portion 34 has a motor 42 mounted to a holding body 41, a gear 43 mounted at a rotating shaft of this motor 42, and a gear 45 mounted at the tip end of a cylindrical body 44 holding the rear end of the assisting instrument insertion portion 31. This gear 45 is meshed with the gear 43 mounted at the rotating shaft of the motor 42. By this, the rotational driving portion 34 can rotate the cylindrical body 44 and the assisting instrument insertion portion 31 by rotating the motor 42 so as to rotate the gear 45.

Also, this motor 42 is connected to a motor control driving portion, not shown, through a cable 46. This motor control driving portion incorporates a battery for driving and a control circuit for controlling revolutions, rotation direction of the motor 42. Also, the motor control driving portion controls/drives the motor 42 of the rotational driving portion 34 according to the operation of the operation knob 10 (See FIG. 1).

By this, the operator can move the assisting instrument insertion portion 31 to the front by tilting the operation knob 10 to the front side. That is, the operator can rotate the motor 42 in the direction to propel the assisting instrument insertion portion 31 by tilting the operation knob 10 to the front side. Also, the operator can move the assisting instrument insertion portion 31 to the rear side by tilting the operation knob 10 to the rear side. That is, the operator can rotate the motor 42 in the direction to move the assisting instrument insertion portion 31 backward by tilting the operation knob 10 to the rear side.

In the rear of the rotational driving portion 34, that is, at the base end portion of the assisting instrument insertion portion 31, the fluid control device 9 is provided. This fluid control device 9 has, as shown in FIGS. 4 and 5, a fluid supply portion 51 provided for supplying a fluid such as air, water and the like to the assisting instrument insertion portion 31.

The fluid supply portion 51 is mounted to a pipeline port 52a of the assisting instrument insertion portion 31 with an O-ring 53 in the air-tight manner. The fluid supply portion 51 supplies the fluid such as air, water and the like from a piping port 55 through a connection tube 54 by a pump, not shown, to the pipeline port 52a of the assisting instrument insertion portion 31 or can discharge the fluid from this pipeline port 52a.

The pipeline port 52a of the assisting instrument insertion portion 31 is formed with notches in the circumferential direction, and even if the assisting instrument insertion portion 31 is rotated, it can communicate with the piping port 55 of the fluid supply portion 51 all the time.

As shown in FIG. 6, at the assisting instrument insertion portion 31, a fluid pipeline 52 for supplying the fluid to the balloon 32 through the pipeline port 52a is disposed. At the tip end side of this fluid pipeline 52, a balloon-side pipeline port 52b opened in the balloon 32 is formed.

In this embodiment, the fluid pipeline 52 is individually provided at each of the balloons 32, and the fluid supply portion 51 is also provided individually according to the fluid pipeline 52.

By this, in the assisting instrument insertion portion 31, the balloon 32 can be inflated/deflated through the fluid pipeline 52 by supplying and discharging the fluid such as air, water and the like by the fluid control device 9. The inflation/deflation of the balloon 32 is executed by the control circuit which controls the pump and the valve control portion through the operation of the fluid control device 9, and the pressure at the inflation is controlled to be a constant pressure.

In this embodiment, excessive deformation of the intracoelomic duct is prevented by locking the assisting instrument insertion portion 31 in the intracoelomic duct through inflation of the balloon 32, and insertion of the endoscope 2 can be smoothly assisted along this assisting instrument insertion portion 31.

Action of insertion of the endoscope 2 into a body cavity using the endoscope insertion assisting device 3 of this embodiment constructed as above will be described. The endoscope insertion assisting device 3 is used under an ultrasonic image by an ultrasonic observing device or an X-ray radiographic image by an X-ray device in order to check the position of the endoscope insertion assisting instrument 7 in the body cavity, though not shown.

FIG. 7 shows insertion from an anus 61 into a deep portion side of a large intestine in the state where the endoscope insertion assisting instrument 7 of the endoscope insertion assisting device 3 is inserted into the treatment instrument channel 22 of the endoscope 2 using the endoscope insertion assisting device 3 of this embodiment in the large intestine.

When the endoscope insertion portion 11 of the endoscope 2 is to be inserted into the large intestine, the operator inserts the endoscope insertion assisting instrument 7 into the treatment instrument channel 22 of the endoscope 2.

First, the operator inserts the endoscope tip end portion 15 into the anus 61. Then, the operator inserts the assisting instrument insertion portion 31 of the endoscope insertion assisting instrument 7 into the treatment instrument insertion port 21 of the endoscope 2, has the tip end portion of the assisting instrument insertion portion 31 protruded from the channel opening 22a of the treatment instrument insertion channel 22 and guides it into a rectum 62.

The endoscope insertion assisting instrument 7 inserted into the rectum 62 has the helical structure portion 33 provided at the assisting instrument insertion portion 31 brought into contact with the large intestine wall. At this time, the contact state between the helical structure portion 33 and the large intestine wall becomes a relation between a male screw and a female screw. In the state where the helical structure portion 33 is in contact with the large intestine wall, the operator operates the operation knob 10 of the rotational driving device 8 so as to drive the motor 42 of the rotational driving portion 34 and to rotate the assisting instrument insertion portion 31.

In the endoscope insertion assisting instrument 7, such a thrust is generated that the male screw is moved to the female screw at the contact portion between the helical structure portion 33 and the large intestine wall, that is, a thrust to propel the assisting instrument insertion portion 31 is generated when the assisting instrument insertion portion 31 is rotated. By this thrust, in the endoscope insertion assisting instrument 7, the assisting instrument insertion portion 31 advances toward the deep portion in the large intestine.

In the endoscope insertion assisting instrument 7, the assisting instrument insertion portion 31 passes from the rectum 62 through a sigmoid colon portion 63 and then, passes through a bent portion, which is a boundary between the sigmoid colon portion 63 and a descending colon portion 64 with little movability, a splenic flexure portion 66, which is a boundary between the descending colon portion 64 and a transverse colon portion 65 with rich movability, and a hepatic flexure portion 67, which is a boundary between the transverse colon portion 65 and an ascending colon portion 68 and reaches the vicinity of a cecum portion 69, which is a target portion, as shown in FIG. 8.

The operator stops operation of the operation knob 10 and stops driving of the motor 42 of the rotational driving portion 34. Next, the operator operates the fluid control device 9 so as to inflate all the balloons 32 of the endoscope insertion assisting instrument 7. In the fluid control device 9, the pump and the valve control portion are driven to supply a fluid such as air, water and the like to the fluid pipeline 52 of the assisting instrument insertion portion 31 through the fluid supply portion 51.

The fluid supplied to the fluid pipeline 52 is transmitted through this fluid pipeline 52 and inflates all the balloons 32 (32a, 32b) as shown in FIG. 9. By this, the endoscope insertion assisting instrument 7 can hold at least a part of the shape of the intracoelomic duct into which the medical device is inserted while being locked in the intracoelomic duct.

In this embodiment, the balloons 32a, 32b are arranged so as to hold the bent portion of the intracoelomic duct. As the balloon 32, only the balloon 32a located particularly on the deep portion side of the bent portion may be provided. By this, the endoscope insertion assisting instrument 7 can suppress deformation of the bent portion of the intracoelomic duct by inflating the balloon 32 and prevent excessive deformation of the intracoelomic duct.

In the state where all the balloons 32 are inflated, the operator carries out the bending operation, pushing operation or twisting operation by the bending operation knob 19 for the endoscope insertion portion 11 of the endoscope 2 and inserts the endoscope insertion portion 11 of the endoscope 2 into the target portion at the deep portion in the body cavity along the assisting instrument insertion portion 31.

First, the operator passes the endoscope tip end portion 15 from the rectum 62 to the sigmoid colon portion 63.

FIG. 10 shows a case where the endoscope tip end portion is passed from the rectum to the sigmoid colon portion using a usual guide wire.

As shown in FIG. 10, using a usual guide wire 70, when the endoscope tip end portion 15 of the endoscope 2 is passed from the rectum 62 to the sigmoid colon portion 63, the endoscope tip end portion 15 is brought into contact with the wall surface of the bent portion of the sigmoid colon portion 63 as shown by a dotted line and might press the wall surface.

In this case, since the usual guide wire 70 is not locked in the intracoelomic duct, the tip end portion pressed between the endoscope tip end portion 15 and the wall surface of the bent portion and having reached the deep portion of the intracoelomic duct is withdrawn into the bent portion. Therefore, with the endoscope 2, it is difficult to insert the endoscope tip end portion 15 into the target portion when using the usual guide wire 70.

However, in this embodiment, the balloons 32 are arranged before and after the bent portion of the sigmoid colon portion 63 as shown in FIG. 11. Thus, the endoscope tip end portion 15 is limited in the progress direction by the assisting instrument insertion portion 31 locked by the balloon 32 in the intracoelomic duct. Also, in the intracoelomic duct, deformation of the bent portion is suppressed by the balloon 32. Therefore, the endoscope tip end portion 15 can pass through the sigmoid colon portion 63 along the assisting instrument insertion portion 31 without going toward the wall surface of the bent portion.

When the endoscope tip end portion 15 reaches immediately before the balloon 32, the operator deflates the balloon 32 immediately before by operating the fluid control device 9. In the fluid control device 9, the pump and the valve control portion are driven and the fluid such as air, water and the like is discharged from the fluid pipeline 52 of the assisting instrument insertion portion 31 through the fluid supply portion 51. By this, the endoscope insertion assisting instrument 7 can deflate the balloon 32 immediately before the endoscope tip end portion 15, and the endoscope tip end portion 15 can pass the bent portion of the sigmoid colon portion 63.

Moreover, when the endoscope tip end portion 15 reaches immediately before the next balloon 32, the operator similarly deflates the next balloon 32 and advances the endoscope tip end portion 15 to the deep portion in the body cavity. In this way, by deflating the balloon 32 immediately before according to the reached position of the endoscope tip end portion 15, the excessive deformation of the intracoelomic duct can be suppressed immediately up to the balloon 32, and the endoscope tip end portion 15 is guided to the target portion where the tip end portion of the assisting instrument insertion portion 31 has reached along the assisting instrument insertion portion 31. By this, the endoscope tip end portion 15 can reach the vicinity of the cecum portion 69, which is the target portion, similarly to the assisting instrument insertion portion 31.

When the endoscope tip end portion 15 has reached the target portion, the operator transfers to pulling back of the endoscope insertion portion 11 in order to perform an endoscopic inspection in the large intestine and carries out the inspection. After the inspection is finished, the operator withdraws the endoscope insertion assisting instrument 7 from the treatment instrument insertion channel 22 of the endoscope 2 and withdraws the endoscope insertion portion 11 of the endoscope 2 from the intracoelomic duct.

As a result, with the endoscope insertion assisting device 3 of this embodiment, by holding at least a part of the shape of the intracoelomic duct into which the endoscope 2 is inserted at insertion of the endoscope 2, excessive deformation of the intracoelomic duct can be suppressed and insertion performance of the endoscope 2 can be improved.

The above first embodiment is constructed so that the pipeline disposed in the assisting instrument insertion portion 31 is provided individually at each balloon 32, but as shown in FIG. 12, it may be so constructed that only a single pipeline is disposed in the assisting instrument insertion portion.

As shown in FIG. 12, an assisting instrument insertion portion 31B is constructed by disposing a common pipeline 71.

The common pipeline 71 has a branch passage 72 branching immediately before the balloon 32. In this branch passage 72, a balloon-side pipeline port 52b opened in the balloon 32 through a control valve 73 arranged in the middle is formed. Also, to the control valve 73, an exhaust passage 74 opened on the outer circumferential face of the assisting instrument insertion portion 31B is connected.

In the control valve 73, a signal line (not shown) is inserted and disposed in the assisting instrument insertion portion 31B, electrically connected to the valve control portion of the fluid control device 9 and opened/closed by control of this valve control portion. When the balloon 32 is to be inflated, the control valve 73 opens/closes so that the balloon-side branch passage 72a communicates with the common pipeline-side branch passage 72b so as to supply the fluid from the common pipeline 71 to the balloon 32 based on an opening/closing signal from the valve control portion.

On the other hand, when the balloon 32 is to be deflated, the control valve 73 opens and closes so that the balloon-side branch passage 72a communicates with the discharge passage 74 so as to discharge the fluid in the balloon 32 to the discharge passage 74 based on the opening and closing signal from the valve control portion. The control valve 73 has a pressure sensor inside, and when the pressure at inflation of the balloon 32 exceeds a set pressure, it opens and closes so that the balloon-side branch passage 72a communicates with the discharge passage 74 and the fluid in the balloon 32 is discharged into the discharge passage 74 to control the pressure at the inflation constant.

By this, since the assisting instrument insertion portion 31B has only one common pipeline 71 disposed, the diameter can be made smaller than a case where pipelines are provided for each balloon, and insertion performance into the intracoelomic duct can be improved.

The assisting instrument insertion portion may be constructed, as shown in FIG. 13, with a sensor provided in the vicinity of the balloon 32 so that the balloon 32 is automatically deflated.

As shown in FIG. 13, the assisting instrument insertion portion 31C has a sensor 75 provided on the outer circumferential face. This sensor 75 is a proximity switch 75A (See FIG. 14) such as a high-frequency oscillator or the like and electrically connected to a control valve 73B. When the endoscope tip end portion 15 is guided and brought closer, the sensor 75 detects (a metal object forming) the endoscope tip end portion 15 (that is, detection of a position of the endoscope, which is a medical device) and outputs a detection signal to the control valve 73. The control valve 73B opens/closes so that the branch passage 72 communicates with the discharge passage 74 and discharges the fluid in the balloon 32 to the discharge passage 74 on the basis of the detection signal from the sensor 75.

By this, in the assisting instrument insertion portion 31C, at insertion of the endoscope insertion portion 11 of the endoscope 2 into the target portion at the deep portion in the body cavity, when the endoscope tip end portion 15 reaches immediately before the balloon 32, the balloon 32 is automatically deflated without operation of the fluid control device 9 by the operator, which further improves operatability. The assisting instrument insertion portion 31C shown in FIG. 13 is constructed using individual pipelines disposed for each balloon 32 similar to the description for the first embodiment, but it may be applied to the common pipeline 71 described in FIG. 12.

Also, the sensor 75 may be constructed as shown in FIGS. 15 to 17, which will be described below. A sensor 75B shown in FIG. 15 is an optical sensor and has a light emitting portion 76a such as an LED and a light receiving portion 76b such as a photo transistor. In the sensor 75B, light emitted from the light emitting portion 76a is reflected by the endoscope tip end portion 15 and this reflected light is detected by the light receiving portion 76b, and a detection signal is outputted.

The sensors 75A, 75B described in FIGS. 14 and 15 are non-contact type switches but they may be contact type switches. Sensors 75C, 75D shown in FIGS. 16, 17 are push-button type switches. These sensors 75C, 75D are turned on when they are brought into contact with the inner wall of the treatment instrument insertion channel 22 and outputs a detection signal upon detection of the endoscope tip end portion 15. The contact type switch may be a snap action switch (also called as a sensitive switch) using an actuator other than the above.

The assisting instrument insertion portion may have the helical structure portion formed by balloons.

An assisting instrument insertion portion 31D is constructed, as shown in FIGS. 18 and 19, with a helical balloon 77 in which a helical structure portion at substantially the same position is formed by a balloon is provided instead of the balloon 32.

The helical balloon 77 has a fluid supplied from the pipeline 52 similarly to the balloon 32. When the assisting instrument insertion portion 31D is inserted to the target portion in the body cavity, the helical balloon 77 is in the deflated state as shown in FIG. 18 and when it reaches the target portion and is locked in the intracoelomic duct, the helical balloon 77 is in the inflated state as shown in FIG. 19.

By this, since it is not necessary to newly provide the balloon 32 in the assisting instrument insertion portion 31D as compared with the assisting instrument insertion portion 31 in the first embodiment, the diameter can be further reduced. The assisting instrument insertion portion may be constructed by combining the helical balloon 77 and the balloon 32.

Also, the assisting instrument insertion portion may be constructed by forming the helical structure portion on the outer circumferential face of the balloon. As shown in FIGS. 20, and 21, the assisting instrument insertion portion 31E is constructed by providing a balloon 32E with a helix provided with a helical structure portion 33E on the outer circumferential face.

When the assisting instrument insertion portion 31E is inserted to the target portion in the body cavity, the balloon 32E with the helix is in the deflated state as shown in FIG. 20, and when it reaches the target portion and is locked in the intracoelomic duct, the balloon 32E with the helix is inflated as shown in FIG. 21.

By this, insertion performance of the assisting instrument insertion portion 31E into the intracoelomic duct is further improved by a portion that the helical structure portion 33E is formed at the balloon portion as compared with the assisting instrument insertion portion 31 in the first embodiment.

Also, the assisting instrument insertion portion may be constructed by providing a balloon on the outer circumference of the helical structure portion.

An assisting instrument insertion portion 31F is constructed, as shown in FIGS. 22, and 23, by providing a balloon 32F on the outer circumference of the helical structure portion 33 so as to contain the helical structure portion 33 inside.

When the assisting instrument insertion portion 31F is inserted to the target portion in the body cavity, the balloon 32F is in the deflated state as shown in FIG. 22, and when it reaches the target portion and is locked in the intracoelomic duct, the balloon 32F is inflated as shown in FIG. 23.

By this, assisting instrument insertion portion 31F obtains the same effect as that of the assisting instrument insertion portion 31E and moreover, there is no irregularity at inflation when the helical structure portion 33F is not formed at the balloon portion, and locking performance in the intracoelomic duct is favorable.

Also, the assisting instrument insertion portion may be constructed by providing a coil spring as locking means (locking portion) instead of the balloon. As shown in FIGS. 24 and 25, an assisting instrument insertion portion 31G is constructed by inserting a coil spring 78 in the helical structure portion 33G and exposing this coil spring 78 at substantially the same position instead of the balloon 32.

When the assisting instrument insertion portion 31G is inserted to the target portion in the body cavity, the coil spring 78 is in the deflated state with the same rotating direction of the coil spring 78 as shown in FIG. 24, and when it reaches the target portion and is locked in the intracoelomic duct, the coil spring 78 is in the inflated state as shown in FIG. 25 by rotating only the coil spring 78 in the reverse direction to release it. The rotation of the coil spring 78 is controlled by a driving mechanism, not shown.

By this, since the assisting instrument insertion portion 31G is constructed only by inserting the coil spring 78 in the helical structure portion 33G, there is no need to incorporate pipelines and the like as compared with the assisting instrument insertion portion 31 in the first embodiment, which allows further reduction of the diameter with a simple construction.

Also, the assisting instrument insertion portion may be constructed by providing suction means (suction portion) as locking means (locking portion) instead of the balloon. As shown in FIG. 26, an assisting instrument insertion portion 31H has a suction pipeline 79 for sucking the inner wall of the body cavity disposed. At the tip end side of this suction pipeline 79, a suction portion 80 is provided having an opening for sucking the inner wall of the body cavity formed.

On the other hand, at the rear end side of this suction pipeline 79, a pipeline connection portion, not shown, with the same structure as that of the fluid supply portion 51 described in the first embodiment is mounted in the air-tight manner and is connected to the pump.

The suction pipeline passage 79 is individually provided at each suction portion 80, and the pipeline connection portion is provided individually according to the suction pipeline 79. By this, the assisting instrument insertion portion 31H can be constructed more easily than the assisting instrument insertion portion 31 in the first embodiment only by providing the suction pipeline 79 without providing the balloon 32, and the diameter can be reduced.

In FIG. 26, the pipelines disposed in the assisting instrument insertion portion 31 are provided individually at each balloon 32, but as shown in FIG. 27, only one pipeline to be disposed in the assisting instrument insertion portion may be provided in the construction.

An assisting instrument insertion portion 31I is constructed by disposing a common suction pipeline 81 as shown in FIG. 27. The common suction pipeline 81 has a branch passage 82 branched immediately before from the suction portion 80. This branch passage 82 is opened in the suction portion 80 through a control valve 83 arranged in the middle. To the control valve 83, an open passage 84 opened on the outer circumferential face of the assisting instrument insertion portion 31B is connected.

The control valve 83 is electrically connected to the valve control portion of the fluid control device 9 through a signal line (not shown) inserted and disposed in the assisting instrument insertion portion 31I, and is opened and closed by control of this valve control portion.

When the inner wall of the body cavity is to be suctioned by the suction portion 80, the control valve 83 opens and closes so that a body cavity side branch passage 82a communicates with a common pipeline side branch passage 82b and the inner wall of the body cavity is suctioned from the common suction pipeline 81 on the basis of the opening and closing signal from the valve control portion. On the other hand, when suction of the inner wall of the body cavity by the suction portion 80 is to be stopped, the control valve 83 opens and closes so that the body cavity side branch passage 82a communicates with the open passage 84 so as to open the body cavity side branch passage 82a on the basis of the opening and closing signal from the valve control portion.

By this, since the assisting instrument insertion portion 31I has only one common suction pipeline 81 disposed, the diameter can be more reduced than the case where the suction pipeline 79 is provided individually at the suction portion 80, and insertion performance into the intracoelomic duct can be improved. The opening/closing operation of the control valve 83 may be so constituted that the suction is stopped immediately before the endoscope tip end portion 15 is brought closer by the detection signal from the sensor 75 for detecting the endoscope tip end portion 15 as described in FIG. 13.

Also, in FIGS. 26 and 27, only one direction of the suction portion 80 is described, but the present invention is not limited to this, but the suction portion 80 may be provided in the both directions or at locations in the circumferential direction of the outer circumferential face of the assisting instrument insertion portion.

Moreover, the assisting instrument insertion portion may be constructed by combining the suction means (suction portion) with the balloon as locking means (locking portion) and providing a suction pipeline at the contact portion between the balloon and the intracoelomic duct (in the vicinity of the balloon center), though not shown. By this, the assisting instrument insertion portion can lock the intracoelomic duct more surely not only by the locking force by the inflation of the balloon but by additional suction force by the suction pipeline.

### SECOND EMBODIMENT

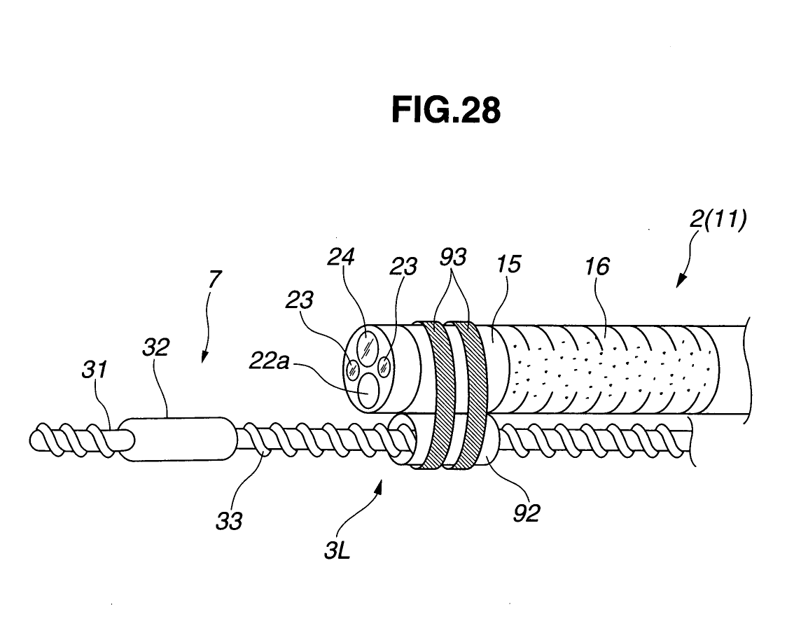

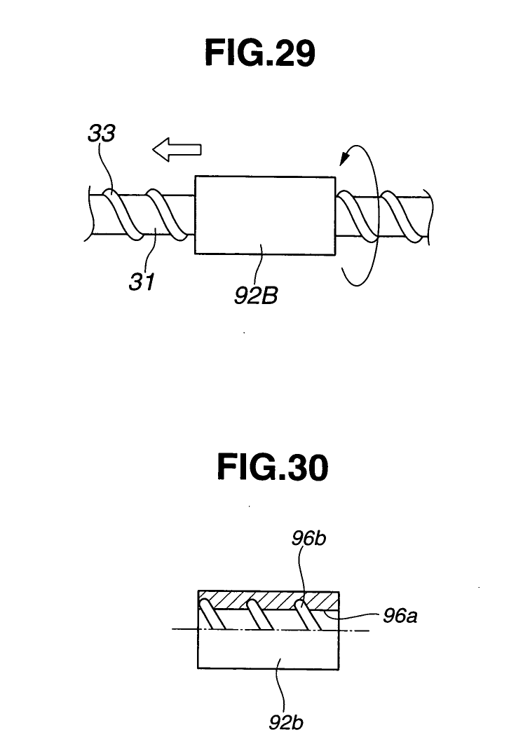

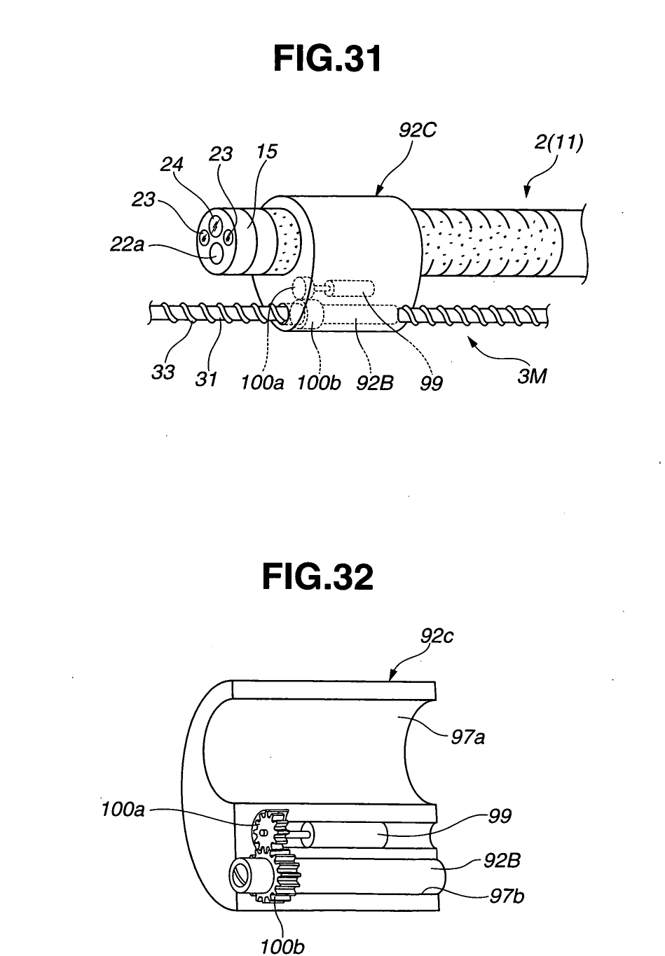

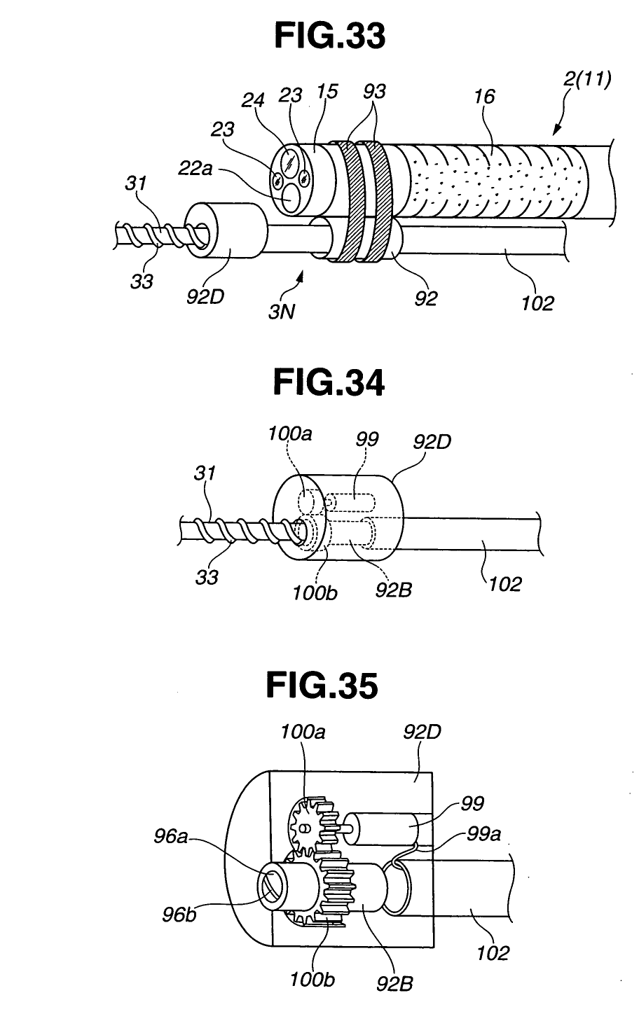

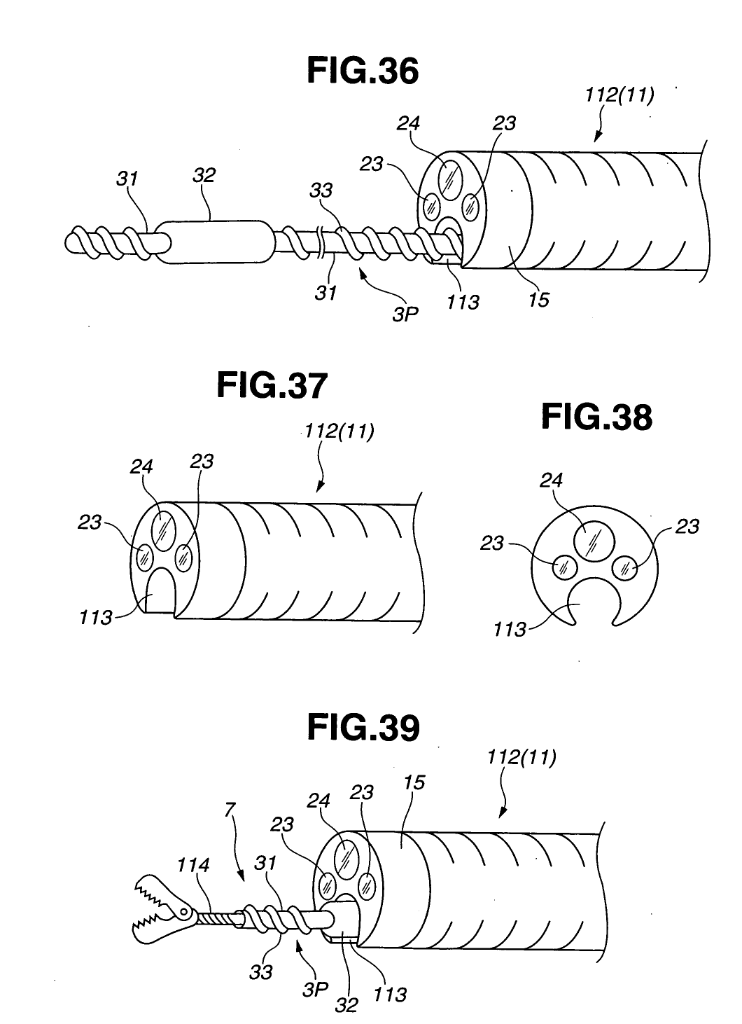

FIGS. 28 to 39 relate to a second embodiment of the present invention, in which FIG. 28 is a perspective view showing a construction of a tip end side of the endoscope device according to a second embodiment, FIG. 29 is a view showing a holding body for propelling in a first variation, FIG. 30 is a view showing a structure of the holding body for propelling in FIG. 29, FIG. 31 is a perspective view showing an outline construction of the holding body for propelling in a second variation, FIG. 32 is a view showing an internal construction of the holding body for propelling in FIG. 31, FIG. 33 is a perspective view showing the vicinity of the holding body for propelling with the endoscope mounted in a third variation, FIG. 34 is a perspective view showing an outline construction of the holding body for propelling in FIG. 33, FIG. 35 is a view showing an internal construction of the holding body for propelling in FIG. 34, FIG. 36 is a perspective view showing a tip end side of a fourth variation inserted into an exclusive endoscope channel, FIG. 37 is a perspective view showing the vicinity of the tip end portion of the endoscope in FIG. 36, FIG. 38 is a front view of the endoscope in FIG. 37, and FIG. 39 is a perspective view showing a state where the treatment instrument is inserted into a hollow portion of a fourth variation.

In the first embodiment, the endoscope insertion assisting instrument 7 is constructed so that the endoscope 2 is guided by inserting the treatment instrument insertion channel 22 of the endoscope 2, but in the second embodiment, the endoscope-insertion assisting instrument 7 is constructed so as to guide this endoscope 2 along the endoscope 2. Since the other constructions are the same as those of the first embodiment, the description is omitted and the same reference numerals are given to the same construction in the description.

As shown in FIG. 28, an endoscope insertion assisting device 3L of the second embodiment is constructed to carry out insertion assistance by being mounted on the outer circumferential face of the endoscope 2. In this endoscope insertion assisting device 3L, a cylindrical body 92 as a holding body for propelling through which the assisting instrument insertion portion 31 of the endoscope insertion assisting instrument 7 can movably pass is fixed to the endoscope tip end portion 15 by a tape 93.

The rear end of the assisting instrument insertion portion 31 is connected to the rotational driving portion 34 of the rotational driving device 8 as with the description in the first embodiment, and by rotating the rear end of the assisting instrument insertion portion 31, the assisting instrument insertion portion 31 can be smoothly propelled.

Moreover, in the rear of the rotational driving portion 34, that is, at the base end portion of the assisting instrument insertion portion 31, the fluid control device 9 is provided as with the description in the first embodiment, and the fluid supply portion 51 is mounted in this fluid control device 9. In the assisting instrument insertion portion 31I, the balloon 32 is inflated/deflated when the fluid such as air, water and the like is supplied and discharged through this fluid supply portion 51.

Action of insertion of the endoscope 2 into the body cavity using the endoscope insertion assisting device 3L of the second embodiment constructed as above will be described. When the endoscope insertion assisting device 3L is used, it is used under an ultrasonic image by an ultrasonic observing device or an X-ray radiographic image by an X-ray device as described in the first embodiment.

First, the operator passes the assisting instrument insertion portion 31 of the endoscope insertion assisting instrument 7 into the cylindrical body 92, and this cylindrical body 92 is fixed to the endoscope tip end portion 15. Next, the operator first inserts the assisting instrument insertion portion 31 of the endoscope insertion assisting instrument 7 protruding to the front from the endoscope tip end portion 15 into a large intestine or the like.

Next, the operator can insert this assisting instrument insertion portion 31 into the deep portion side in the body cavity such as a large intestine or the like by smooth propelling by rotating the rear end of the assisting instrument insertion portion 31 by the rotational driving mechanism. Since the subsequent operation is the same as that of the first embodiment, the description will be omitted.

According to this embodiment, it can be also used for the endoscope 2 provided with the endoscope insertion portion 11 with a small diameter not having the treatment instrument insertion channel 22 and can be used for insertion assistance of the endoscope 2.

The holding body for propelling may be constructed as shown in FIGS. 29 to 32.

As shown in FIGS. 29 and 30, a holding body 92C for propelling has a hole 96a through which the assisting instrument insertion portion 31 is passed and a nut-shaped guide 92B formed with a pitch of the helical structure portion 33 provided on the outer circumferential face of the assisting instrument insertion portion 31 and having a helical groove 96b for housing this helical structure portion 33.

As shown in FIG. 31, in the holding body for propelling 92C, a hole 97a through which the vicinity of the endoscope tip end portion 15, for example, of the endoscope insertion portion 11 of the endoscope 2 is passed is formed as shown in a cut-away view of FIG. 32, and a hole 97b is formed for holding the nut-shaped guide 92B through which the assisting instrument insertion portion 31 provided with the helical structure portion 33 is passed.

Also, inside this holding body for propelling 92C, a motor 99 for rotational driving is provided. A gear 100a mounted to a rotating shaft of this motor 99 is meshed with a gear 100b mounted to the outer circumferential face of the nut-shaped guide 92B. The holding body for propelling 92C portion around the gears 100a, 100b is cut away so that the gears 100a, 100b can rotate. The motor 99 is connected to a motor control device, not shown, by a signal line, and rotation and rotation stop of the motor 99 can be controlled by operating the operation knob provided at this motor control device.

In this way, the holding body for propelling 92C is constructed for movable rotation control of the assisting instrument insertion portion 31.

Also, the operator can rotate and drive the nut-shaped guide 92B by rotationally driving the motor 99 by operating the operation knob. On the inner circumferential face of the nut-shaped guide 92B, a hole through which the assisting instrument insertion portion 31 is passed as described in FIG. 30 and a helical groove with which the helical structure portion 33 is fitted and passed are provided.

According to this first variation, in the endoscope insertion assisting device constructed as above, the endoscope tip end portion 15 can be propelled along the assisting instrument insertion portion 31 by rotating the motor 99 for rotational driving mounted to the holding body for propelling 92C after the assisting instrument insertion portion 31 has been inserted into the body cavity such as a large intestine. As a result, the endoscope insertion assisting device can propel the endoscope insertion portion 11 constituting the medical device into the deep portion of the intracoelomic duct more efficiently.

The holding body for propelling may be constructed as shown in FIGS. 33 to 35.

As shown in FIG. 33, the endoscope insertion assisting device 3L has a sheath 102 through which the assisting instrument insertion portion 31 is inserted. At the tip end of this sheath 102, a holding body for propelling 92D is provided.

This holding body for propelling 92D is shown in FIG. 34. Also, FIG. 35 shows the internal structure of the holding body for propelling 92D. This holding body for propelling 92D has substantially the same structure as that of the holding body for propelling 92C shown in FIG. 32. That is, as shown in FIG. 35, the motor 99 for rotational driving, the gear 100a mounted to the rotating shaft of this motor 99, the gear 100b meshed with this gear 100a and the nut-shaped guide 92B on which this gear 100b is mounted are provided inside the holding body for propelling 92D.

After insertion of the assisting instrument insertion portion 31 into the deep portion in the body cavity, the operator operates the operation knob.

In the holding body for propelling 92D, by rotational driving of the motor 99, the nut-shaped guide 92B rotatably held inside the holding body for propelling 92D is rotationally driven so as to propel the sheath 102 to the tip end portion of the assisting instrument insertion portion 31.

In this variation, since the assisting instrument insertion portion 31 with the helical structure portion 33 provided on the outer circumferential face is covered by the sheath 102 with the flat outer circumferential face, an effect is exerted that insertion work of the endoscope 2 is made smooth.

The endoscope device may be constructed as shown in FIGS. 36 to 39.

As shown in FIGS. 36 to 38, the endoscope device is constructed by providing an exclusive endoscope 112 into which the assisting instrument insertion portion 31 of an endoscope insertion assisting instrument 7P is inserted.

In this variation, the exclusive endoscope 112 having a tip-end opening 113 (and a channel with the same sectional shape as that of this tip-end opening 113) capable of detachable insertion from below, for example, may be used so that the assisting instrument insertion-portion 31 of the endoscope insertion assisting instrument 7P is made to protrude to the front from this tip-end opening 113 to be used for insertion assistance. This endoscope 112 has the same construction in the endoscope insertion portion 11 and the other portions as that of the above-mentioned endoscope 2.

In the case of this variation, the endoscope insertion assisting instrument 7P can be used as the guide wire 70. Also, in the case of this endoscope insertion assisting instrument 7P, a treatment can be performed by forming a hollow portion in the assisting instrument insertion portion 31 as shown in FIG. 39 and by inserting a treatment instrument 114 into this hollow portion. Moreover, the endoscope device, not shown, can be used by inserting the endoscope insertion assisting device into the channel of the endoscope for treatment instrument having a large-diameter channel and a plurality of channels from the endoscope tip end.

### INDUSTRIAL APPLICABILITY

Since the insertion assisting instrument for a medical device of the present invention can improve insertion performance of the medical device into the deep portion in the intracoelomic duct by holding at least a part of the shape of the intracoelomic duct into which the medical device is to be inserted to suppress excessive deformation of the intracoelomic duct at insertion into the deep portion of the intracoelomic duct, it is suitable for inspection, observation, treatment and the like in a subject.

1-13. (canceled)

14. An insertion assisting instrument for a medical device, comprising:
an elongated insertion portion having flexibility for guiding the medical device into a deep portion in an intracoelomic duct;
a helical structure portion provided on the outer circumference of the insertion portion; and
a plurality of locking portions for holding at least a part of the shape of the intracoelomic duct into which the medical device is inserted and for locking the insertion portion in the intracoelomic duct.

15. The insertion assisting instrument for the medical device according to claim 14, wherein the locking portion is constructed capable of inflation/deflation in the radial direction of the insertion portion and locks the insertion portion in the intracoelomic duct by being inflated in the radial direction of the insertion portion and pressing an inner wall of the intracoelomic duct.

16. The insertion assisting instrument for the medical device according to claim 14, wherein the locking portion has a suction portion capable of suction of the inner wall of the intracoelomic duct and locks the insertion portion in the intracoelomic duct by sucking the inner wall of the intracoelomic duct by this suction portion.

17. The insertion assisting instrument for the medical device according to claim 15, wherein the helical structure portion also functions as the locking portion.

18. The insertion assisting instrument for the medical device according to claim 14, wherein a control portion for controlling the locking portion is provided.

19. The insertion assisting instrument for the medical device according to claim 15, wherein a control portion for controlling the locking portion is provided.

20. The insertion assisting instrument for the medical device according to claim 16, wherein a control portion for controlling the locking portion is provided.

21. The insertion assisting instrument for the medical device according to claim 17, wherein a control portion for controlling the locking portion is provided.

22. The insertion assisting instrument for the medical device according to claim 14, wherein the locking portion is a balloon.

23. The insertion assisting instrument for the medical device according to claim 15, wherein the locking portion is a balloon.

24. The insertion assisting instrument for the medical device according to claim 16, wherein the locking portion is a balloon.

25. The insertion assisting instrument for the medical device according to claim 17, wherein the locking portion is a balloon.

26. The insertion assisting instrument for the medical device according to claim 18, wherein the locking portion is a balloon.

27. The insertion assisting instrument for the medical device according to claim 22, wherein at least five balloons are arranged at the insertion portion.

28. The insertion assisting instrument for the medical device according to claim 22, wherein the control portion has a valve for controlling supply/discharge of a fluid.

29. The insertion assisting instrument for the medical device according to claim 15, wherein the locking portion is a coil spring.

30. The insertion assisting instrument for the medical device according to claim 29, wherein the control portion has a driving mechanism for controlling rotation of the coil spring.

31. The insertion assisting instrument for the medical device according to claim 18, wherein the control portion has a valve for controlling a suction pressure of the suction portion.

32. The insertion assisting instrument for the medical device according to claim 14, wherein the insertion portion is provided with a sensor for detecting the position of the medical device.

33. The insertion assisting instrument for the medical device according to claim 32, wherein the control portion controls the locking portion according to a detection result of the sensor, holds at least a part of the shape of the intracoelomic duct into which the medical device is inserted and locks the insertion portion in the intracoelomic duct.
